(19) United States
(12) Patent Application Publication
(10) Pub. No.: US 2006/0182729 A1
(43) Pub. Date: Aug. 17, 2006

(54) Combat/training antioxidant micronutrient formulation and method of administration

(76) Inventors: Kedar N. Prasad, Novato, CA (US); Gerald M. Haase, Greenwood Village, CO (US); William C. Cole, Novato, CA (US)

Correspondence Address: OSTROLENK FABER GERB & SOFFEN, 1180 AVENUE OF THE AMERICAS, NEW YORK, NY, 100368403, US

(21) Appl. No.: 11/357,676

(22) Filed: Feb. 17, 2006

Related U.S. Application Data
(60) Provisional application No. 60/653,909, filed on Feb. 17, 2005.

(51) Int. Cl. A61K 31/714; A61K 38/43; A61K 31/525; A61K 31/51; A61K 31/455; A61K 31/4415

(52) U.S. Cl. 424/94.1; 424/641; 424/702; 514/52; 514/168; 514/251; 514/276; 514/350; 514/356; 514/440; 514/393; 514/458; 514/474; 514/574

(57) ABSTRACT

The invention is directed to a method for administering antioxidant micronutrient formulations comprising certain multiple dietary and endogenous antioxidants, B-vitamins, vitamin D, and minerals at appropriate doses and dose-schedules, particularly chosen to reduce acute and long-term adverse effects of environmental and physical stressors and toxic chemicals in Operating Forces, e.g., military troops, during training and in combat. The invention additionally encompasses a variety of antioxidant micronutrient formulations that are useful in reducing, if not entirely eliminating, acute and/or chronic damage in Operating Forces produced by free radicals and products of inflammatory reactions that are generated by the environmental and physical stressors and toxic chemicals to which such Operating Forces may be exposed.

### CROSS-REFERENCE TO RELATED APPLICATION

This application claims the priority of Provisional Application No. 60/653,909 filed Feb. 17, 2005, the contents of which are specifically incorporated by reference herein.

### BACKGROUND OF THE INVENTION

1. Field of the Invention

The invention is directed to a method for administering antioxidant micronutrient formulations comprising certain multiple dietary and endogenous antioxidants, B-vitamins, vitamin D, and minerals at appropriate doses and dose-schedules, particularly chosen to reduce acute and long-term adverse effects of environmental and physical stressors, and toxic chemicals in Operating Forces, e.g., military troops, during training and in combat. The invention additionally encompasses a variety of antioxidant micronutrient formulations that are useful in reducing, if not entirely eliminating, acute and/or chronic damage in Operating Forces produced by free radicals and products of inflammatory reactions that are generated by the environmental and physical stressors, and toxic chemicals to which such Operating Forces are exposed.

2. Description of the Related Art

Operating Forces are exposed to extreme environmental conditions including heat, cold, dehydration, dust particles, high repeated noise waves, physical exhaustion and toxic solvents such as diesel fuel and hydrocarbons during training. These agents are known to generate increased amounts of free radicals derived from oxygen and nitrogen that can increase oxidative stress, leading to adverse acute health effects such as increased frequency of colds, physical exhaustion and overall not feeling well in Operating Forces.

In combat, Operating Forces are additionally exposed to blast exposures that are generated by explosive devices such as bombs, mortars, artillery shells, missiles, anti-tank weapons and land mines. Blast exposures have become one of the major causes of injuries in Operating Forces in combat. Blast exposures cause damage by generating excessive amounts of inorganic and organic free radicals and by the products of inflammatory reactions that include pro-inflammatory cytokines, prostaglandins and other toxic chemicals. Free radicals, reactive oxygen species and pro-inflammatory cytokines can produce acute adverse health and increase the risk of chronic disease such as cancer, cataract, heart disease, and neurological diseases.

In combat, Operating Forces also may be exposed to chemical warfare agents that include major classes of chemicals such as mustard agents (sulfur mustards and nitrogen mustards), nerve agents (Tabum, Sarin, Soman, GF and VX), ricin and chlorine gas. These chemical toxins also produce excessive amounts of different types of free radicals and acute inflammatory reactions.

Although a number of patents and published U.S. patent applications describe a variety of nutrition supplements containing herbs, fruit and vegetable extracts, or one or more antioxidant components, no multiple antioxidant micronutrient supplements containing both dietary and endogenous antioxidants have yet been developed, i.e, prior to the present invention, that are adapted for at least reducing, if not entirely eliminating, the damage in Operating Forces who are exposed to environmental, training and combat-related stressors such as those identified above.

For example, U.S. Pat. No. 5,976,568 to Riley focuses on reducing the risk of heart disease in which oxidative damage pays an important role. The reference describes seven distinct modules that include varying levels of antioxidants, in addition to combinations of herbs with antioxidants, as well as with aspirin. In addition, the reference also discloses the administration of iron, copper and manganese. The inclusion of these minerals with vitamin C, however, may be detrimental to the health of Operating Forces since each of the minerals interacts with vitamin C and would thereby produce excessive amounts of free radicals in the bodies of such Operating Forces.

In addition, these trace minerals are more readily absorbed from the intestinal tract when such antioxidants are present, as opposed to when they are absent. This could lead to an increase in iron, copper and manganese stores in the body, since nature has provided no significant mechanisms of excretion of these trace minerals among men of all ages and women after menopause. The resultant increased iron store, for example, can increase the risk of chronic diseases including cancer.

Thus, the nutrition supplement proposed in the '568 Riley patent may reduce the efficacy of antioxidants in reducing the risk of stressors that affect Operating Forces. Additionally, selecting the dose of an ingredient from the range provided by the reference is very difficult. For example, selecting 2,000 mg of Vitamin E from Example 2 could produce a clotting defect following long term consumption. In addition, certain herbs are known to interact with prescription and non-prescription drugs in an adverse manner; therefore, inclusion of herbs in reducing the adverse effects of stressors in Operating Forces may not be suitable. Furthermore, it has been reported that alpha tocopheryl succinate is the most effective form of vitamin E. Alpha tocopherol succinate is not, however, included in any of the modules described by the reference. In summary, therefore, administration of the formulations proposed in the '568 Riley patent to Operating Forces is not an effective strategy for reducing or eliminating damage produced by environmental and combat-related stressors.

U.S. Pat. No. 5,292,538 to Paul et al. provides a series of formulations, as well as a dose range for each of the ingredients of the formulations, wherein the formulations include iron, copper and manganese. The limitations of these iron, copper and manganese-containing products are the same as described above in the discussion of the '568 Riley patent. In addition, some of the formulations described in the reference contain heavy metals such as vanadium and molybdenum, which are known to be neurotoxic; and therefore, can adversely affect the health of Operating forces following long-term consumption.

U.S. Pat. No. 6,805,880 to Hojgaare et al. is directed to a slow-release formulation of vitamin C (ascorbic acid) and a plain-release formulation of vitamin E (tocopherol). The preparation appears to increase the plasma level of these antioxidants. These investigators have focused on cardiovascular disease. The most important protective agent in the cells is glutathione. Glutathione can not be taken orally, because it is totally hydrolyzed in the intestine. Glutathione-elevating agents such as alpha-lipoic acid, N-acetylcysteine and selenomethionine are very strong protective agents, yet they are not included in the formulations described in the reference. Therefore, these formulations remain of limited value for reducing damage produced by environmental and combat stressors in Operating Forces.

U.S. Pat. No. 6,090,414 to Passwater describes a series of formulations that contain vitamin C, butylated hydroxytoulene (BHT), selenomethionine and methionine, or vitamin C, vitamin E, butylated hydroxytoulene (BHT), selenomethionine, cysteine, lecithin and vitamin B12, or vitamin C, vitamin E, butylated hydroxytoulene (BHT), selenomethionine, methionine, lecithin and vitamin B12, or casein, glucose, salt mix with vitamin mix containing B-vitamins, vitamin D and only tocopherol and vitamin A as antioxidants. However, the lack of glutathione-elevating agents such as alpha-lipoic acid and n-acetylcysteine in the formulations described in this reference may reduce their effectiveness in reducing oxidative damage, if they are able to do so at all. Furthermore, the dose and dose—schedules that are so important in reducing damage by antioxidants have not been given adequate attention by the inventors of the '414 patent.

Published U.S. Patent Application. No. US 2003/0105027 of Rosenbloom describes a mixture of antioxidants that includes vitamin A, vitamin D3, beta-carotene, vitamin E, alpha-lipoic acid, quercetin, ascorby plamitate, curcumin, green tea, chlorophyllin, carboxyl ethyl sesquioxide of germanium and superoxide dismutase (SOD) for radiation protection. Other well-established, important antioxidants such as selenomethionine, co-enzyme Q10, N-actylcysteine, are missing from the proposed formulations, however. Additionally, Alpha tocopheryl succinate, the most effective form of vitamin E, is not present in the formulation. Therefore, the formulations disclosed in the reference may not be optimally effective in reducing damage produced by increased free radicals. Further, the addition of germanium, a heavy metal, that could produce neurotoxicity after a long-term consumption, limits the formulations' value in reducing the chronic effect of oxidative damage. In addition, the reference includes no consideration of an appropriate dose-schedule, which is very important in determining the efficacy of antioxidant in reducing oxidative damage. Further, it is not certain whether the formulations described in the subject reference are relevant to damage produced by acute inflammatory agents such as those which are likely to be encountered by Operating Forces in training and/or combat environments.

U.S. Pat. No. 4,619,829 to Motschan is directed to a series of nutritional supplements. However, one such formulation described by the reference contains only an antioxidant and vitamin C, and another formulation has only three antioxidants, vitamin A, vitamin C and vitamin E. A vitamin dose of 25,000 ITJ, such as is taught for use by the reference, can induce birth defects in the fetuses of pregnant women. Moreover, vitamin C and vitamin E—doses of 150 mg and 10 mg., respectively, as disclosed in the reference, are too low to be effective against high levels of environmental and combat-related stressors facing Operating Forces. Furthermore, other well-established important antioxidants such as selenomethionine, co-enzyme Q10, N-acetylcysteine and alpha-lipoic acid, are entirely missing from the disclosed formulations. In addition Alpha tocopheryl succinate, the most effective form of vitamin E, is not included in any of the formulations described in the reference.

Still further, U.S. Pat. No. 5,922704 to Bland describes formulations for 5 tablets and one soft gel capsule to be used to promote ‘optimal health’, but it does not address the health issues facing Operational Forces during training and in combat. The formulations have very low levels of N-acetylcysteine that is not sufficient to increase the intracellular levels of glutathione, and have no alpha-lipoic acid or Co-enzyme Q10, which are endogenous antioxidants necessary to reduce oxidative damage in Operating Forces produced by environmental and combat-related stressors. These formulations also include no alpha-tocopheryl succinate, the most effective form of vitamin E. They do, however, include heavy metals such as molybdenum and minerals such as copper and manganese that interact with vitamin C to produce increased amounts of free radicals. Therefore the formulations described in the reference are not suitable for reducing oxidative damage and inflammatory reactions in Operating Forces.

U.S. Pat. No. 6,245,360 B1 describes supplements developed to correct a nutritional deficiency associated with an addiction to alcohol. The formulation is not, however, adequate for reducing oxidative damage to Operating Forces during training or in combat.

U.S. Pat. No. 6,291,533 B1 to Feischner describes a dietary supplement developed to be responsive to specific blood types, and thus most beneficial for people with specific antigenic blood types. Again, however, the formulation is not adequate for reducing oxidative damage to Operating Forces during training or in combat.

U.S. Pat. No. 6,660,293 B2 to Giordano describes a formulation that was developed for prophylactic nutritional supplementation and therapeutic nutritional supplementation. The formulation (See, e.g., Table 1) lacks important antioxidants such as vitamin A, coenzyme Q10 and N-acetylcysteine. It also lacks alpha-tocopheryl succinate, the most effective form of vitamin E. Furthermore, it includes copper and manganese, both of which interact with vitamin C and which thereby produce increased amounts of free radicals. Therefore, the formulation described in the reference is not adequate for reducing oxidative damage to Operating Forces during training or in combat.

U.S. Pat. No. 6,579,544 B1 to Rosenberg et al. describes a dietary supplement, the composition of which is based on a person's age, body weight and quality of diet. The supplement does not address the health issues facing Operational Forces during training and/or in combat, however. The formulations include all antioxidants, including many fruit and vegetable extracts, without providing any scientific rationale for why these materials reduce oxidative damage. Furthermore, some of the extracts are not well defined and may interact with prescription and non-prescription drugs in an adverse manner. In addition, the formulations include iron, copper and manganese which, as noted above, interact with vitamin C to produce increased amounts of free radicals. Therefore, the formulation is not adequate for reducing oxidative damage to Operating Forces during training or in combat.

U.S. Pat. No. 6,573,299 B1 to Petrus describes an antioxidant and mineral formulation that was specifically developed for the treatment of an aging eye. However, the formulation disclosed by the reference is not adequate for reducing oxidative damage to Operating Forces during training or in combat.

Published U.S. Patent Application No. 2003/0108624 A1 of Kosbab describes a series of formulations for the prevention and treatment of chronic diseases and disorders, including the complications of diabetes mellitus. It does not, however, address the health issues facing Operational Forces during training or in combat. The formulations described in the reference have listed all of the antioxidants, including many fruit and vegetable extracts as well as herbal extracts, without any scientific rationale for their reducing oxidative damage. In addition, some of the extracts are not well defined and may interact with prescription and non-prescription drugs in an adverse manner. Furthermore, the disclosed formulations further contain heavy metals such as cadmium and strontium that are considered toxic, and minerals such as manganese that interact with vitamin C to produce increased amounts of free radicals. Therefore the indicated formulations are not suitable for reducing oxidative damage and inflammatory reactions in Operating Forces during training and in combat.

Published U.S. Patent Application No. 2002/0182196 A1 of McCleary describes formulations developed for normalizing impaired or deteriorating function in humans. The formulations described in the reference can also maximize insulin and glucose metabolism. However, they do not address the health issues facing Operational Forces during training or in combat. The formulations of the reference include many antioxidants, including many fruit and vegetable extracts and herbal extracts, without any scientific rationale for their reducing oxidative damage. Furthermore, some of the extracts are not well defined and may interact with the prescription and non-prescription drugs in an adverse manner. Additionally, the formulations of the reference contain no antioxidants such as vitamin A and N-acetylcysteine. They additionally lack alpha-tocopheryl succinate, which is the most effective form of vitamin E. Therefore the disclosed formulations are also not suitable for reducing oxidative damage and inflammatory reactions in Operating Forces during training and in combat.

Published U.S. Patent Application No. 2004/0082536 A1 of Cooper et al. describes formulations of multiple vitamin and mineral supplements for use by men, post-menopausal and pre-menopausal women, and athletes, which supply the proper amounts of the correct micronutrients needed for disease prevention and protection against nutritional losses. However, the formulations described by the reference do not address the health issues facing Operational Forces during training or in combat. For example, these formulations have no important antioxidants such as glutathione-elevating agents, alpha-lipoic acid and N-acetylcysteine. They, additionally, lack alpha-tocopheryl succinate, the most effective form of vitamin E. Further, they contain iron and copper that interact with vitamin C and produce increased amounts of free radicals. Therefore the formulations described by the reference are not suitable for reducing oxidative damage and inflammatory reactions in Operating Forces during training and in combat.

Published U.S. Patent Application No. 2002/0146463 A1 of Clayton provides a series of formulations for improving health by treating, preventing, or curing diseases and medical disorders. However, these formulations do not address the health issues facing Operational Forces during training or in combat. The formulations have listed a number of antioxidants, including many fruit and vegetable extracts and herbal extracts, without any scientific rationale for their reducing oxidative damage. Additionally, some of the extracts are not well defined and may interact with prescription and non-prescription drugs in an adverse manner. Still further, these formulations lack glutathione-elevating agents such as alpha-lipoic acid and N-acetylcysteine. They also lack alpha-tocopheryl succinate, the most effective form of vitamin E. Further, they contain manganese and copper that interact with vitamin C to produce increased amounts of free radicals. The formulations additionally include the toxic heavy metal, molybdenum. Some formulations contain high doses of insulin that could be harmful. Therefore the formulations described in the reference are not suitable for reducing oxidative damage and inflammatory reactions in Operating Forces during training and in combat.

Published U.S. Patent Application No. 2002/0193323 A1 of Inna describes a formulation for promoting a healthy cardiovascular system and enhancing blood flow. It does not, however, address the health issues facing Operational Forces during training or in combat. The formulations described by the reference contains certain herbal extracts without providing any scientific rationale for their reducing oxidative damage. Furthermore, these extracts are not well defined and may interact with the prescription and non-prescription drugs in an adverse manner. The formulations additionally lack the glutathione-elevating agent, N-acetylcysteine, as well as alpha-tocopheryl succinate, the most effective form of vitamin E. The described formulations, moreover, contain manganese that interacts with vitamin C and produces increased amounts of free radicals. It also contains glutathione that is totally destroyed in the small intestine, and thus, has no value, together with glutamic acid that, at high doses, is considered neurotoxic. Therefore the proposed formulation is not suitable for reducing oxidative damage and inflammatory reactions in Operating Forces during training and in combat.

There has thus been a long-felt need in the prior art for formulations and methods adapted for administering such formulations, comprising certain multiple dietary and endogenous antioxidants, B vitamins, vitamin D and certain minerals at appropriate dosages and dose schedules to reduce acute and long-term effects of environmental and physical stressors and toxic chemicals typically encountered by Operating Forces during training and in combat.

### SUMMARY OF THE INVENTION

The invention thus provides a method for administering formulations comprising certain multiple dietary antioxidants and their derivatives (vitamin A, vitamin C, alpha-tocopheryl acetate, alpha-tocopheryl succinate, selenium as selenomethionine), and endogenous antioxidants (alpha lipoic acid, coenzyme Q10, and a glutathione-elevating agent, n-acetylcysteine), B-vitamins, vitamin D, and certain minerals, but no iron, copper or manganese, at appropriate doses and dose-schedules to reduce acute and long-term adverse effects of environmental and physical stressors and toxic chemicals in Operating Forces during training and in combat. The operating forces are exposed to extreme environmental conditions including heat, cold, dehydration, dust particles, high repeated noise waves, physical exhaustion and toxic solvents during training. In combat, they are additionally exposed to blast exposures that are generated by explosive devices such as bombs, mortars, artillery shells, missiles, anti-tank weapons and land mines. In addition, they could be exposed to chemical weapons that include major classes of chemicals such as mustard agents (sulfur mustards and nitrogen mustards), nerve agents (Tabum, Sarin, Soman, GF and VX), ricin and chlorine gas.

Many of above agents have specific mechanisms of action, but they have some common mechanisms of producing damage. These mechanisms involve production of excessive amounts of different types of free radicals and acute inflammatory reactions. Reactive oxygen species (ROS) and pro-inflammatory cytokines such as interleukin-6 (IL-6) and tumor necrosis factor-α (TNF-α) are released during inflammatory reaction. Free radicals, reactive oxygen species and pro-inflammatory cytokines can produce acute adverse health and increase the risk of chronic disease such as cancer, cataract, heart disease, and neurological diseases. As indicated above, until the present invention no multiple antioxidant micronutrient supplements containing dietary and endogenous antioxidants that could reduce damage produced by environmental, training and combat-related stressors in Operating Forces, have been developed.

It is, therefore, an object of the present invention to provide formulations, dosages and dose-schedules of the same, that are capable of reducing, if not entirely eliminating, acute and chronic damage in Operating Forces produced by free radicals and products of inflammatory reactions that are generated by diverse groups of agents. Data obtained following testing of the formulations of the invention such as proposed here on civilians clearly demonstrate that the formulations, dosage and dose schedule described herein are very important in reducing oxidative stress and stimulating immune functions. Many prior art formulations are not capable of providing such protection due, at least in part, to the fact that they lack appropriate ingredients or teachings regarding appropriate dosages and/or dose schedules. Moreover, various animal studies demonstrate that a mixture of antioxidants, all of which are present in the formulation of the present invention, protected the animals against damage produced by radiation-induced free radicals as well as from acute inflammatory reactions.

It is a further object of the invention to provide formulations, dosages and dose-schedules that are also capable of enhancing the rate of wound healing after surgery. With regard to this aspect of the invention, published studies have shown that antioxidants are protective against ischemia and re-perfusion injury and that they enhance wound healing. Enhanced beneficial results, moreover, are obtainable through administration of formulations in accordance with those of the present invention.

Furthermore, high levels of toxic agents (mutagens and carcinogens) are formed during digestion of food. Since “Meals Ready to Eat” (MREs) as supplied by the military have been designed to contain low fiber, the increased bowel emptying time for food could lead to increased exposure to these toxins. This could have adverse long-term health consequences because increased amounts of mutagens may remain for a longer time within the bowel, leading to increased adsorption of these toxins. It is, therefore, a further object of the invention to provide formulations, dosages and dose-schedules which serve to reduce the levels of toxins that are formed during digestion of MREs. In support, studies have suggested that supplementation with vitamin C and vitamin E reduces the formation of mutagens in the small intestine; and further, the combination of these vitamins was more effective than the results obtained with the administration of each such agent individually.

In one embodiment, the invention is directed to an antioxidant micronutrient formulation comprising:

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

Alternately, in a further embodiment the invention is directed to a more specific antioxidant micronutrient formulation comprising:

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

In an additional embodiment, the invention is directed to a method for providing an antioxidant micronutrient formulation to a member of an Operating Force to reduce acute or long-term adverse effects of at least one of environmental and physical stressors and toxic chemicals to which the Operating Force member may be subjected during training or in combat, wherein the method comprises administering to the Operating Force member, during training or in combat, at least once per day, an antioxidant micronutrient formulation as shown below. In one embodiment of the invention, the formulation below is administered twice per day. The formulation comprises

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

A further embodiment comprises a method for reducing the levels of toxins that are formed in the gastrointestinal (G.I.) tract of a member of an Operating Force upon the digestion of a Meal Ready to Eat (MRE) supplied to the Operating Force member, wherein the method comprises administering to each such member ingesting such a MRE, at least once per day, an antioxidant micronutrient formulation as shown below. In an embodiment of the invention, the formulation is administered twice per day. The formulation comprises

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

Another embodiment of the invention relates to a method of enhancing the rate of wound healing after surgery on a member of an Operating Force which comprises administering one or more times to the Operating Force member, following such surgery, an antioxidant micronutrient formulation which comprises

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

### DESCRIPTION OF CERTAIN PREFERRED EMBODIMENTS

As noted above, it has been determined that the administration to Operating Forces of multiple dietary and endogenously formed antioxidants will serve to reduce the harmful effects associated with oxidative stress, e.g., by reducing oxidative damage and increasing immune functions. As used herein, the term “Operating Forces” includes, but is not necessarily limited to, military troops. That is, the term should be functionally defined to include any individual who may be exposed to stressors and toxic materials of the type described herein, whether or not they are members of a military organization, although such exposure is typically more common in the case of military troops.

Without wishing to be bound by theory, it is believed that many different types of free radicals are produced, and each antioxidant has a different mechanism of action and a different affinity for each of these free radicals, depending upon the cellular environment. In addition, the various antioxidants are synergistic with each other in order to increase their efficacy to protect against oxidative damage produced by free radicals.

Vitamin C is necessary to protect cellular components in aqueous environments, whereas carotenoids, i.e., vitamins A and E, protect cellular components in lipid environments. Vitamin C also plays an important role in maintaining cellular levels of vitamin E by recycling the vitamin E radical (oxidized) to the reduced (antioxidant) form. The form and type of vitamin E used are also important for a maximal effect in reducing oxidative damage caused by free radicals. Various organ tissues selectively absorb the natural form of vitamin E. Alpha tocopherol succinate is the most effective form of the vitamin in reducing growth of cancer cells.

Selenium is a co-factor of glutathione peroxidase, and Se-glutathione peroxidase also acts as an antioxidant. Therefore, selenium supplementation of the antioxidants taught herein for inclusion in the formulations of the invention is also essential for maximally reducing oxidative damage.

Glutathione, an endogenously formed compound, represents a potent intracellular protective agent against damage produced by free radicals. It catabolizes H₂O₂ and anions. Oral supplementation with glutathione failed to significantly increase plasma levels of glutathione in human subjects, suggesting that this tripeptide is completely hydrolyzed in the G.I. tract. However, N-acetylcysteine and alpha-lipoic acid can effectively increase the intracellular levels of glutathione by different mechanisms.

Although coenzyme Q10 is a weak antioxidant, it acts as a co-factor for generating ATP in the mitochondria. It also scavenges peroxy radicals faster than alpha-tocopherol and, like vitamin C, can regenerate vitamin E in a redox cycle.

In an exemplary embodiment, the formulations of the invention may be supplied in hard gelatin capsules. The ingredients remain stable at high temperatures (up to 50° C. for 3 days) and cold temperature (up to −20° C. for 3 days) due to the protection provided by the relatively hard gelatin capsules.

In one embodiment, the invention provides an antioxidant micronutrient formulation comprising:

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

In a further, exemplary, embodiment of the invention, the formulation may comprise the following components in the following specific amounts:

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

In another embodiment, the invention is directed to a method for providing an antioxidant micronutrient formulation to a member of an Opposing Force to reduce or substantially eliminate acute or long-term effects of at least one of environmental and physical stressors and toxic chemicals to which the Operating Force member may be subjected during training or in combat. The method comprises administering to such Operating Force members, during training or in combat, at least once per day an antioxidant micronutrient formulation as shown below. In an embodiment of the invention, the formulation is administered twice per day. The formulation comprises

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

In an example embodiment of the above-described method, the antioxidant micronutrient formulation of the invention may be administered to the Operating Force member at least two times per day. In another embodiment, the antioxidant micronutrient formulation is administered to the Operating Force member via a capsule (e.g., a gelatin capsule or a softsule) at least two times per day wherein, at each administration, the Operating Force member is provided with at least two capsules or softsules containing the formulation and wherein each administrations take place within about 12 hours from the previous administration. In a further embodiment, the administration may be timed to coincide with the Operating Force member's morning and evening meals.

In a further alternate embodiment of the invention, the formulation provided to the Operating Force member may comprise

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

In an example embodiment of the above-described method, the antioxidant micronutrient formulation of the invention may be administered to the Operating Force member at least two times per day. In another embodiment, the antioxidant micronutrient formulation is administered to the Operating Force member via a capsule (e.g., a gelatin capsule or a softsule) at least two times per day wherein, at each administration, the Operating Force member is provided with at least two capsules or softsules containing the formulation and wherein each administrations take place within about 12 hours from the previous administration. In a further embodiment, the administration may be timed to coincide with the Operating Force member's morning and evening meals.

In a further embodiment, the invention is directed to a method for reducing the levels of toxins that are formed in the gastrointestinal (G.I.) tract of a member of an Operating Force due to digestion of a Meal Ready to Eat (MRE) supplied to the Operating Force member. The method comprises administering to each such Operating Force member ingesting such a MRE, at least once (e.g., twice) per day, an antioxidant micronutrient formulation comprising

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

In an example embodiment of the above-described method, the antioxidant micronutrient formulation of the invention may be administered to the Operating Force member at least two times per day. In another embodiment, the antioxidant micronutrient formulation is administered to the Operating Force member via a capsule (e.g., a gelatin capsule or a softsule) at least two times per day wherein, at each administration, the Operating Force member is provided with at least two capsules or softsules containing the formulation and wherein each administrations take place within about 12 hours from the previous administration. In a further embodiment, the administration may be timed to coincide with the Operating Force member's morning and evening meals.

In a further alternate embodiment of the invention, the formulation provided to the Operating Force member may comprise

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

In an example embodiment of the above-described method, the antioxidant micronutrient formulation of the invention may be administered to the Operating Force member at least two times per day. In another embodiment, the antioxidant micronutrient formulation is administered to the Operating Force member via a capsule (e.g., a gelatin capsule or a softsule) at least two times per day wherein, at each administration, the Operating Force member is provided with at least two capsules or softsules containing the formulation and wherein each administrations take place within about 12 hours from the previous administration. In a further embodiment, the administration may be timed to coincide with the Operating Force member's morning and evening meals.

In still another embodiment, the invention is directed to a method of enhancing the rate of wound healing after surgery on a member of an Operating Force which comprises administering to the Operating Force member, following such surgery, an antioxidant micronutrient formulation which comprises

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

In an example embodiment of the above-described method, the antioxidant micronutrient formulation of the invention may be administered to the Operating Force member at least two times per day. In another embodiment, the antioxidant micronutrient formulation is administered to the Operating Force member via a capsule (e.g., a gelatin capsule or a softsule) at least two times per day wherein, at each administration, the Operating Force member is provided with at least two capsules or softsules containing the formulation and wherein each administrations take place within about 12 hours from the previous administration. In a further embodiment, the administration may be timed to coincide with the Operating Force member's morning and evening meals.

In a further alternate embodiment of the invention, the formulation provided to the Operating Force member may comprise

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

In an example embodiment of the above-described method, the antioxidant micronutrient formulation of the invention may be administered to the Operating Force member at least two times per day. In another embodiment, the antioxidant micronutrient formulation is administered to the Operating Force member via a capsule (e.g., a gelatin capsule or a softsule) at least two times per day wherein, at each administration, the Operating Force member is provided with at least two capsules or softsules containing the formulation and wherein each administrations take place within about 12 hours from the previous administration. In a further embodiment, the administration may be timed to coincide with the Operating Force member's morning and evening meals.

Although the present invention has been described in relation to particular embodiments thereof, many other variations and modifications and other uses will become apparent to those skilled in the art. The present invention, therefore, is not limited by the specific disclosure herein, but only by the claims.

What is claimed is:

1. An antioxidant micronutrient formulation comprising:

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

2. The antioxidant micronutrient formulation of claim 1, wherein the formulation comprises:

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

3. A method for providing an antioxidant micronutrient formulation to a member of an Operating Force to reduce acute or long-term adverse effects of at least one of environmental and physical stressors and toxic chemicals to which said Operating Force member may be subjected during training or in combat, the method comprising administering to said Operating Force member, during training or in combat, at least once per day, an antioxidant micronutrient formulation which comprises:

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

4. The method according to claim 3, wherein the antioxidant micronutrient formulations administered to said operating force member at least two times per day.

5. The method according to claim 4, wherein the antioxidant micronutrient formulation, is administered to said Operating Force member via a capsule or a softsule containing said formulation and wherein at least two said capsules or softsules are administered about twelve hours apart on a daily basis while said Operating Force member is in training or in combat.

6. The method according to claim 3, wherein the antioxidant micronutrient formulation is administered to said Operating Force member with, respectively, the member's morning and evening meals.

7. The method of claim 3, wherein said antioxidant micronutrient formulation comprises:

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

8. The method according to claim 7, wherein the antioxidant micronutrient formulation is administered to said operating force member at least two times per day.

9. The method according to claim 8, wherein the antioxidant micronutrient formulation, is administered to said Operating Force member via a capsule or a softsule containing said formulation and wherein at least two said capsules or softsules are administered about twelve hours apart on a daily basis while said Operating Force member is in training or in combat.

10. The method according to claim 7, wherein the antioxidant micronutrient formulation is administered to said Operating Force member with, respectively, the member's morning and evening meals.

11. A method for reducing the levels of toxins that are formed in the gastrointestinal (G.I.) tract of a member of an Operating Force due to the digestion of a Meal Ready to Eat (MRE) as supplied to said Operating Force member, which comprises administering to each said member ingesting said MRE's, at least once a day, an antioxidant micronutrient formulation comprising:

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

12. The method according to claim 11, wherein the antioxidant micronutrient formulation is administered to said operating force member at least two times per day.

13. The method according to claim 12, wherein the antioxidant micronutrient formulation, is administered to said Operating Force member via a capsule or a softsule containing said formulation and wherein at least two said capsules or softsules are administered about twelve hours apart on a daily basis while said Operating Force member is in training or in combat.

14. The method according to claim 11, wherein the antioxidant micronutrient formulation is administered to said Operating Force member with, respectively, the member's morning and evening meals.

15. The method of claim 11, wherein the antioxidant micronutrient formulation comprises:

| | Total Dose/day | |
|---|---|---|
| Vitamin A (as retinyl palmitate) | 5,000 | IU |
| Vitamin C (as calcium ascorbate) | 1,000 | mg |
| Vitamin E (as d-alpha-tocopherol) | 200 | IU |
| (as d-alpha-tocopheryl succinate) | 200 | IU |
| Vitamin D (as cholecalciferol) | 400 | IU |
| Vitamin B-1 (thiamine mononitrate) | 4 | mg |
| Vitamin B-2 (riboflavin) | 5 | mg |
| Niacin (as niacinamide ascorbate) | 30 | mg |
| Vitamin B-6 (as pyrodioxine HCl) | 5 | mg |
| Folate (folic acid) | 800 | mcg |
| Vitamin B-12 (as cyanocobalamin) | 10 | mcg |
| Biotin | 200 | mcg |
| Pantothenic acid (as calcium pantothenate) | 10 | mg |
| Calcium citrate | 250 | mg |
| Magnesium citrate | 125 | mg |
| Zinc (as zinc glycinate) | 15 | mg |
| Selenium (as L-selenomethionine) | 100 | mcg |
| Chromium (as chromium piconilate) | 50 | mcg |
| Coenzyme Q10 | 30 | mg |
| n-acetylcysteine | 250 | mg |
| Alpha-lipoic acid | 60 | mg |

16. The method according to claim 15, wherein the antioxidant micronutrient formulation is administered to said operating force member at least two times per day.

17. The method according to claim 16, wherein the antioxidant micronutrient formulation, is administered to said Operating Force member via a capsule or a softsule containing said formulation and wherein at least two said capsules or softsules are administered about twelve hours apart on a daily basis while said Operating Force member is in training or in combat.

18. The method according to claim 15, wherein the antioxidant micronutrient formulation is administered to said Operating Force member with, respectively, the member's morning and evening meals.

19. A method of enhancing the rate of wound healing after surgery on a member of an Operating Force which comprises administering to said Operating Force member, following said surgery, an antioxidant micronutrient formulation which comprises:

| | Total Dose/day | |
|---|---|---|
| Vitamin A (as retinyl palmitate) | 3,000-5,000 | IU |
| Vitamin C (as calcium ascorbate) | 100-4,000 | mg |
| Vitamin E (as d-alpha-tocopherol) | 50-400 | IU |
| (as d-alpha-tocopheryl succinate) | 50-400 | IU |
| Vitamin D (as cholecalciferol) | 400-600 | IU |
| Vitamin B-1 (thiamine mononitrate) | 2-10 | mg |
| Vitamin B-2 (riboflavin) | 2-20 | mg |
| Niacin (as niacinamide ascorbate) | 15-200 | mg |
| Vitamin B-6 (as pyrodioxine HCl) | 2-10 | mg |
| Folate (folic acid) | 400-1,200 | mcg |
| Vitamin B-12 (as cyanocobalamin) | 5-20 | mcg |
| Biotin | 100-500 | mcg |
| Pantothenic acid (as calcium pantothenate) | 5-30 | mg |
| Calcium citrate | 100-500 | mg |
| Magnesium citrate | 100-250 | mg |
| Zinc (as zinc glycinate) | 10-30 | mg |
| Selenium (as L-selenomethionine) | 50-200 | mcg |
| Chromium (as chromium piconilate) | 50-200 | mcg |
| Coenzyme Q10 | 10-250 | mg |
| n-acetylcysteine | 100-500 | mg |
| Alpha-lipoic acid | 15-100 | mg |

20. The method according to claim 19, wherein the antioxidant micronutrient formulation is administered to said operating force member at least two times per day.

21. The method according to claim 20, wherein the antioxidant micronutrient formulation, is administered to said Operating Force member via a capsule or a softsule containing said formulation and wherein at least two said capsules or softsules are administered about twelve hours apart on a daily basis following said surgery.

22. The method according to claim 19, wherein the antioxidant micronutrient formulation is administered to said Operating Force member with, respectively, the member's morning and evening meals.
